(19) United States
(12) Patent Application Publication
(10) Pub. No.: US 2007/0249528 A1
(43) Pub. Date: Oct. 25, 2007

(54) Histamine Binding Compounds for Treatment Method for Disease Conditions Mediated by Neutrophils

(76) Inventor: Wynne Weston-Davies, Berkshire (GB)

Correspondence Address: Klauber & Jackson, 411 Hackensack Avenue, 4th Floor, Hackensack, NJ, 07601, US

(21) Appl. No.: 10/551,482

(22) PCT Filed: Apr. 1, 2004
(86) PCT No.: PCT/GB04/01428

§ 371 Date: Jul. 31, 2006

(30) Foreign Application Priority Data
Apr. 1, 2003 (GB) 0307544.7

(51) Int. Cl. A61K 38/02; A61P 29/00; A61P 37/00

(52) U.S. Cl. 514/12

(57) ABSTRACT

The invention relates to a novel method for the treatment of disease conditions that are mediated by neutrophil cells. The method involves the administration to a patient suffering from such a condition, a histamine binding compound in a therapeutically-effective amount.

The invention relates to a novel method for the treatment of disease conditions that are mediated by neutrophil cells. The method involves the administration to a patient suffering from such a condition, a histamine binding compound in a therapeutically-effective amount.

All publications, patents and patent applications cited herein are incorporated in full by reference.

Many inflammatory and auto-immune conditions are characterised by the influx of neutrophils to the site of disease. In some cases this influx is inappropriate and causes damage to normal tissue.

In many types of neutrophil-mediated disease tissue injury is thought to be associated with oxidative free radicals released by activated neutrophils (Dahlgren C, Karlsson A. Respiratory burst in human neutrophils. J Immunol Methods Dec. 17, 1999;232(1-2):3-14) and tissue myeloperoxidase (MPO) activity may be used to quantify this.

Examples of disease conditions mediated by neutrophils include adult respiratory distress syndrome (ARDS); infant respiratory distress syndrome (IRDS); severe acute respiratory syndrome (SARS); chronic obstructive airways disease (COPD); cystic fibrosis; ventilator induced lung injury (VILI); capillary leak syndrome; reperfusion injury including injury following thrombotic stroke, coronary thrombosis, cardiopulmonary bypass (CPB), coronary artery bypass graft (CABG), limb or digit replantation, organ transplantation, bypass enteritis, bypass arthritis, thermal injury and crush injury; post-operative inflammation or marginal infiltrates, psoriasis; psoriatic arthropathy; rheumatoid arthritis; Crohn's disease; ulcerative colitis; immune vasculitis including Wegener's granulomatosis and Churg-Strauss disease; alcoholic liver disease; neutrophil mediated glomerulonephritis; systemic lupus erythematosus; lupus nephritis; atherosclerosis; systemic sclerosis; gout; periodontal disease, ocular inflammation including dry eye, Sjogren's syndrome, contact lens associated papillary conjunctivitis (CLAPC), contact lens associated marginal infiltrates, post surgical inflammation including surgery for cataract, glaucoma, corneal transplantation and laser in-situ keratomileusis (LASIK), severe allergic conjunctivitis, vernal keratoconjunctivitis (VKC), diffuse lamellar keratitis, infective and non-specific conjunctivitis, keratitis and blepharitis, and shield ulcers.

Although histamine has been known to be involved in virtually all allergic and inflammatory processes it has not previously been implicated as having any role in neutrophil mediated disease. Certain antihistamine agents have been tested for utility in counteracting diseases of this nature, but these have been agents that target histamine receptors, rather than targeting histamine itself. Furthermore, even when used in combination therapy with other pharmaceuticals, such agents have been of limited use when tested in animal models of endotoxin-induced lung damage (Byrne K, Sielaff T D, Michna B, Carey P D, Blocher C R, Vasquez A, Sugerman H J. Crit Care Med. March 1990;18(3):303-8; Byrne K, Sielaff T D, Carey P D, Tatum J L, Blocher C R, Vasquez A, Hirsh J I, Sugerman H J, Circ Shock. February 1990;30(2):117-27; Sielaff T D, Sugerman H J, Tatum J L, Kellum J M, Blocher C R., J Trauma. December 1987;27(12):1313-22; Sielaff T D, Sugerman H J, Tatum J L, Blocher C R., Surgery. August 1987;102(2):350-7). Human treatment protocols do not include histamine blocking agents to treat conditions of this nature (Bernard G R, Artigas A, Brigham K L, Carlet J, Falke K, Hudson L, Lamy M, Legall J R, Morris A, Spragg R. Am J Respir Crit Care Med March 1994;149(3 Pt 1):818-24).

Neutrophil-mediated diseases are a significant health problem and are associated with significant morbidity and mortality. Present methods for targeting these conditions fall well short of being effective. The inventors have now found that these disease conditions can be treated very effectively using agents that bind directly to histamine and thus titrate this vasoactive amine out of the system.

Accordingly, the present invention provides a method of treating a disease condition mediated by neutrophil cells in a patient, comprising administering a histamine binding compound to the patient in a therapeutically-effective amount.

The inventors' discovery is that by completely removing histamine from a disease site, neutrophil-mediated disease conditions may be counteracted. This is only possible using an agent that binds with high affinity to histamine, which explains in part why the effect of histamine on these conditions has not previously been identified; histamine binding agents of this type have only recently been discovered and are not in widespread use. Although previous research has explored a potential role for histamine by using agents that bind to histamine receptors, only a marginal effect was noted. With hindsight, the failure to influence the tested conditions was probably because of the variety of histamine receptors that exist (H1, H2, H3, H4 as well as possible other receptors not yet discovered). Consequently, by targeting the histamine receptors rather than the histamine molecule itself, the agents used were ineffective and thus histamine's role in these conditions has been overlooked.

Neutrophil cells are produced and matured in the bone marrow and migrate from this tissue to their site of action. Once they reach this point, their normal role is to act to destroy pathogenic invading organisms that have been marked for removal by processes such as opsonisation or the complement system. They accomplish this by the release of cytotoxic oxidative free radicals and by phagocytosis. They also remove damaged tissue cells that have undergone apoptosis. It is when they are attracted by a quantitatively or qualitatively inappropriate chemoattractant signal that they may attack normal cells and provoke the damage characteristic of neutrophil mediated disease. It is our contention that histamine may be critical in provoking such an inappropriate chemoattractant signal to be generated.

Neutrophils are known to express histamine receptors (Wescott S, Kaliner M., Inflammation September 1983;7(3):291-300; Burde R, Seifert R, Buschauer A, Schultz G., Naunyn Schmiedebergs Arch Pharmacol December 1989;340(6):671-8). However, it is unlikely that histamine plays a significant role in the mobilisation of neutrophils from their site of production and maturation in the bone marrow since this compound is metabolised and removed from circulation very rapidly (Ferreira S H, Ng K K, Vane J R., Br J Pharmacol. November 1973;49(3):543-53). Although the inventors do not wish to be bound by any particular theory, it is thought more likely that histamine might be acting indirectly through a variety of other mechanisms which attract neutrophils to the site of disease and which are themselves known to be at least partially histamine dependent. These may include inter alia: expression of adhesion molecules by vascular endothelial cells (Jones D A, Abbassi O, McIntire L V, McEver R P, Smith C W., Biophys J October 1993;65(4):1560-9), inhibition of chemokine cytokine-induced neutrophil chemoattractant (Harris J G, Flower R J, Watanabe K, Tsurufuji S, Wolitzky B A, Perretti M., Biochem Biophys Res Commun Apr. 25, 1996;221(3):692-6), release of LTB4 (Takeshita K, Sakai K, Bacon K B, Gantner F. J Pharmacol Exp Ther. December 2003;307(3):1072-8) and release of IL-16 by T lymphocytes (Gantner F, Sakai K, Tusche M W, Cruikshank W W, Center D M, Bacon K B, J Pharmacol Exp Ther. October 2002;303(1):300-7). It has previously been demonstrated that these activities are mediated through different histamine receptors and that there is considerable overlap so that, for instance, it is likely that IL-16 release is controlled both by H₂ and H₄ receptors. It is also possible that further histamine receptors remain to be identified.

Furthermore there is now evidence for a critical role of histamine in the expression of L-selectin adhesion molecules by vascular endothelium and the histamine H4 receptor in the zymosan-induced mobilisation of neutrophils from the bone marrow (Takeshita K, Bacon K B, Gantner F. J Pharmacol Exp Ther. Mar. 2, 2004 [Epub ahead of print]). Together these recent data support the role of histamine acting through a number of receptors, but most critically the H4 receptor, in the recruitment and activation of neutrophils and their involvement in various models of human disease.

With this degree of redundancy and promiscuity in the system it is unlikely that blockade of a single histamine receptor type will prevent the recruitment of neutrophils and this may be one reason for the apparent failure of histamine antagonists tested so far. In contrast, compounds that scavenge free histamine will prevent this agent from reaching any of its receptors, including those that have not yet been discovered. This property contributes to its efficacy as a useful therapeutic agent.

A number of disease conditions are mediated by neutrophils, including allergic, inflammatory and auto-immune conditions. In particular, neutrophil-mediated disease conditions of note include adult respiratory distress syndrome (ARDS); infant respiratory distress syndrome (IRDS); severe acute respiratory syndrome (SARS); chronic obstructive airways disease (COPD); cystic fibrosis; ventilator induced lung injury (VILI); capillary leak syndrome; reperfusion injury including but not limited to injury following thrombotic stroke, coronary thrombosis, cardiopulmonary bypass (CPB), coronary artery bypass graft (CABG), limb or digit replantation, organ transplantation, bypass enteritis, bypass arthritis, thermal injury and crush injury; post-operative inflammation or marginal infiltrates, psoriasis; psoriatic arthropathy; rheumatoid arthritis; Crohn's disease; ulcerative colitis; immune vasculitis including but not limited to Wegener's granulomatosis and Churg-Strauss disease; alcoholic liver disease; neutrophil mediated glomerulonephritis; systemic lupus erythematosus; lupus nephritis; atherosclerosis; systemic sclerosis; gout; periodontal disease, ocular inflammation including dry eye, Sjogren's syndrome, contact lens associated papillary conjunctivitis (CLAPC), contact lens associated marginal infiltrates, post surgical inflammation including surgery for cataract, glaucoma, corneal transplantation and laser in-situ keratomileusis (LASIK), severe allergic conjunctivitis, vernal keratoconjunctivitis (VKC), diffuse lamellar keratitis, infective and non-specific conjunctivitis, keratitis and blepharitis, and shield ulcers. Other neutrophil-mediated conditions will be known to those of skill in the art. Any one of these conditions may be treated in accordance with the present invention.

The histamine binding compound used in the method of the invention should act as a histamine scavenger, that binds to the histamine molecule and thus titrates it out of the system. Such a scavenger thus “mops up” systemic histamine that is present at the site of disease or injury.

The histamine binding compound should preferably bind to histamine with an affinity of at least 10⁻⁵M, more preferably less than 10⁻⁶M, less than 10⁻⁷M, less than 10⁻⁸M, less than 10⁻⁹M, less than 10⁻¹⁰M or less. A suitable histamine binding assay that allows the affinity of a test compound for histamine to be tested is given in International patent application WO97/44451.

The histamine binding compound should preferably be specific for vasoactive amines, in particular histamine. Methods for measuring specificity will be known to those of skill in the art and include competition assays and the like. Preferably, the affinity for histamine displayed by the histamine binding compound is 100-fold greater than that exhibited for unrelated compounds, more preferably, 10³-fold, 10⁴-fold, 10⁵-fold, 10⁶-fold or greater.

The histamine binding compound used in the present invention may be a synthetic compound, or a natural compound such as a protein. A number of proteins are known that exhibit specific high affinity binding to histamine. One possibility is to use antibodies specific for histamine, or antibody fragments. Preferred proteins are the compounds referred to as vasoactive amine binding molecules in International patent application WO97/44451, the contents of which are incorporated herein in their entirety. The term “vasoactive amine binding molecules” is intended to encompass:
(a) any vasoactive amine binding protein that binds specifically to histamine with a dissociation constant of less than 10⁻⁷M and which belongs to the same protein family as the proteins MS-HBP1, FS-HBP1 and FS-HBP-2 disclosed in International Patent Application No. WO97/44451, wherein a protein is considered to belong to this protein family if the primary, mature monomer sequence of the protein has no more than 260 amino acids and at least 30 of the amino acids in the protein's complete sequence are conserved as identical residues in an alignment of that protein and the proteins MS-HBP1, FS-HBP1 and PS-HBP-2, the alignment preferably having been obtained using ClustalW (Thompson et al., 1994, NAR, 22(22), 4673-4680) or a similar sequence alignment program;
(b) a protein from a haematophagous arthropod that binds specifically to histamine with a dissociation constant less than 10⁻⁷M and which contains the sequence motifs D/E A W K/R (preferably DAWK, more preferably QDAWK) and Y/C E/D L/I/P W (preferably Y/C ELW);
(c) a natural biological variant, such as an allelic variant or a geographical variant, of a protein as defined in (a) or (b) above;
(d) a functional equivalent of a protein as defined in (a), (b) or (c) above that contains single or multiple amino-acid substitution(s), addition(s), insertion(s) and/or deletion(s) from the wild type protein sequence and/or substitutions of chemically-modified amino acids that do not affect the biological function of binding to histamine;
(e) an active fragment of a protein as defined in (a), (b), (c) or (d) above, wherein “active fragment” denotes a truncated protein that retains the biological function of binding to histamine; and
(f) a fusion protein comprising a protein as defined in (a), (b), (c), (d) or (e) above fused to a peptide or other protein, such as a label, which may be, for instance, bioactive, radioactive, enzymatic or fluorescent, or an antibody.

Particularly preferred is the protein referred to in WO97/44451 as FS-HBP2 (also known as EV131), or a variant or an active fragment thereof as recited in (a), (b), (c), (d) or (e) above. This protein binds to histamine with high affinity and specificity and is shown herein to be effective in an animal model of neutrophil-mediated disease.

Active fragments according to (e) above should comprise at least n consecutive amino acids from the sequence of the protein responsible for binding to histamine and, depending on the particular sequence, n preferably is 7 or more (for example, 8, 10, 12, 14, 16, 18, 20, 50, 100, 150, 200, 250 or more). Such fragments may be “free-standing”, i.e. not part of or fused to other amino acids or polypeptides, or they may be comprised within a larger polypeptide of which they form a part or region. When comprised within a larger polypeptide, the fragment of the invention most preferably forms a single continuous region. Additionally, several fragments may be comprised within a single larger polypeptide.

Histamine binding proteins for use in the invention may be prepared in recombinant form by expression of their encoding nucleic acid molecules in vectors contained within a host cell. Such expression methods are well known to those of skill in the art and many are described in detail by Sambrook et al (supra) and Fernandez & Hoeffler (1998, eds. “Gene expression systems. Using nature for the art of expression”. Academic Press, San Diego, London, Boston, New York, Sydney, Tokyo, Toronto). The coding sequences for the vasoactive amine binding proteins mentioned above are set out in International patent application WO97/44451. Methods for the production of these molecules, including suitable vectors, host cells and methods for purification of the proteins are also described in this patent application.

The histamine binding compounds may be formulated into pharmaceutical compositions, presented, for example, in unit-dose or multi-dose containers. For example, sealed ampoules and vials and may be stored in a freeze-dried condition requiring only the addition of the sterile liquid carrier immediately prior to use. The dosage will depend on the specific activity of the histamine binding compound and can be readily determined by routine experimentation.

A pharmaceutical composition may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The histamine binding compounds or pharmaceutical compositions utilised in this invention may be administered by any number of routes including, but not limited to, oral, intravenous, intramuscular, intra-arterial, intramedullary, intrathecal, intraventricular, transdermal or transcutaneous applications (for example, see WO98/20734), subcutaneous, intraperitoneal, intranasal, enteral, topical, sublingual, intravaginal or rectal means. In the case of histamine binding proteins, since proteins may be broken down in the stomach, these proteins are preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation isotonic with the blood of the recipient, and aqueous and non-aqueous sterile suspensions which may include suspending agents or thickening agents.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

The term “therapeutically effective amount” as used herein refers to an amount of histamine binding compound needed to treat, ameliorate, or prevent the targeted neutrophil-mediated disease condition, or to exhibit a detectable therapeutic or preventative effect. For any compound, the therapeutically effective dose can be estimated initially either in cell culture assays, for example, of neoplastic cells, or in animal models, usually mice, rabbits, dogs, or pigs. The animal model may also be used to determine the appropriate concentration range and route of administration. Such information can then be used to determine useful doses and routes for administration in humans.

The precise effective amount for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician. Generally, an effective dose will be from 0.005 mg/kg to 50 mg/kg, preferably 0.125 mg/kg to 20 mg/kg. For example, particularly preferred dosages of vasoactive amine binding molecules such as EV131 and EV504 referred to herein as between 0.1 to 20 mg/kg, more preferably, 0.5 to 10 mg/kg, still more preferably 1 to 2 mg/kg. Compositions may be administered individually to a patient or may be administered in combination with other agents, drugs or hormones.

Gene therapy may be employed to effect the endogenous production of a histamine binding protein by specific cells in a patient. Gene therapy can either occur in vivo or ex vivo. Ex vivo gene therapy requires the isolation and purification of patient cells, the introduction of the therapeutic gene and introduction of the genetically altered cells back into the patient. In contrast, in vivo gene therapy does not require isolation and purification of a patient's cells.

The therapeutic gene is typically “packaged” for administration to a patient. Gene delivery vehicles may be non-viral, such as liposomes, or replication-deficient viruses, such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. For example, a nucleic acid molecule encoding a histamine binding protein may be engineered for expression in a replication-defective retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo (see Chapter 20, Gene Therapy and other Molecular Genetic-based Therapeutic Approaches, (and references cited therein) in Human Molecular Genetics (1996), T Strachan and A P Read, BIOS Scientific Publishers Ltd).

Another approach is the administration of “naked DNA” in which the therapeutic histamine binding compound is directly injected into the bloodstream or muscle tissue.

According to a still further aspect of the invention there is provided the use of a histamine binding compound as recited in any one of the aspects of the invention described above, in the manufacture of a medicament for the treatment of a disease condition mediated by neutrophil cells, particularly those diseases explicitly recited herein.

The invention will now be described by way of example, with explicit reference to the use of the EV131 protein in an experimental model of endotoxin-induced ARDS in mice. It will be appreciated that modification of detail may be made without departing from the scope of the invention.

### BRIEF DESCRIPTION OF THE FIGURES

FIG. 1: Endotoxin (LPS) induced bronchoconstriction and inhibition by EV131. LPS was given at 1 mg by the intranasal route and EV131 at 360 μg, 180 μg and 90 μg. PenH values were measured for 3 h. At 3 h the response to methacholine was analysed. The codes S01, S02 etc. each represent an individual mouse (souris).

FIG. 2: EV131 inhibits endotoxin-induced neutrophil recruitment in BAL. LPS was given at 1 μg by the intranasal route and EV 131 at 360 μg, 180 μg and 90 μg. Total cells did not differ, while EV131 180 μg and 90 μg reduced the neutrophils in BAL.

FIG. 3: EV131 inhibits endotoxin-induced neutrophil recruitment in lung as assessed by MPO activity. EV131 at 180 μg and 90 μg, but not at 360 μg inhibited MPO activity in the lungs.

FIG. 4: Endotoxin (LPS) induced bronchoconstriction and inhibition by EV131 as administered intraperitoneally by injection. LPS was given at 1 μg by the intranasal route and EV131 at 182 μg. PenH values were measured for 3 h.

FIG. 5: Total cell recruitment in BAL fluid when rEV131 and budenoside are given intraperitoneally.

FIG. 6: Cell recruitment in BAL fluid when rEV131 and budenoside are given intraperitoneally, as differentiated by cell type.

FIG. 7: Total cell recruitment in BAL fluid when rEV131 and budenoside are given intraperitoneally.

FIG. 8: TNF in the BAL fluid is reduced by rEV131 as given intraperitoneally.

FIG. 9: Schematic diagram for the intradermal injection sites 1-8 in the back of the skin. 1,2 negative controls (saline); 7,8 positive controls (anti-Ova); 3-6 Inhibition of Ova effects by EV proteins (decreasing concentrations) coadministered with the anti-Ova serum injected intradermally.

FIG. 10: Inhibition of vascular leakage by rEV131 in WB/ReJ C57B1/6j-kit w mice (w/w).

FIG. 11: Spectrophotometric quantification of immune complex mediated vascular leakage. Dose dependence of the inhibitory effects of EV131 and EV504.

FIG. 12: PMN infiltration at the site of the Arthus reaction. Microscopic investigation of injection site at 6 h. Negative control (Saline; A); Positive reaction (Anti-Ova; B); Total inhibition by EV131 (Anti-Ova+EV131; C); and partial inhibition by EV504 (Anti-Ova+EV504; D).

FIG. 13: Neutrophil counts in tear samples suggest that unpreserved rEV131 significantly decreases the number of neutrophils recruited to the eye during or immediately after conjunctival allergen challenge in human patients.

### EXAMPLE 1

### Allergic Asthma

The recombinant, arthropod derived histamine binding protein EV131 binds histamine with high affinity (Paesen, G. C., P. L. Adams, K. Harlos, P. A. Nuttall, and D. I. Stuart. 1999, Mol Cell 3:661; Paesen, G. C., P. L. Adams, P. A. Nuttall, and D. L. Stuart. 2000, Biochim Biophys Acta 1482:92).

Initially, tests were performed to ascertain whether EV131 might inhibit pathologies mediated by histamine. EV131 was therefore tested in allergic asthma. EV131 given prior to antigen challenge in immunised mice was found to prevent airway hyperreactivity by 70%, abrogated peribronchial inflammation, pulmonary eosinophilia, mucus hypersecretion and IL-4 secretion (Couilllin et al, submitted). The inhibitory effect of EV131 on bronchial hyperreactivity was comparable to that of glucocorticosteroids. These results demonstrate that histamine is a critical mediator of allergic asthma.

The results of these tests prompted us to investigate acute respiratory distress syndrome (ARDS), which shows certain features in common with allergic asthma. A model for ARDS was established using a single administration of E. coli endotoxin (Lefort, J., L. Motreff, and B. B. Vargaftig. 2001, Am J Respir Cell Mol Biol 24:345.). It is here shown that EV131 dramatically inhibits bronchoconstriction and neutrophil recruitment.

Methods

Induction of Acute Bronchoconstriction by E. coli Endotoxin

The optimal dose of endotoxin that would produce maximal airways responses without killing the mice was first established using saline alone as control. This was determined to be 1 μg (data not shown). E. coli endotoxin (055:B5, Sigma) was dissolved in saline and given to C57BL/6 mice at a dose of 1 μg in 40 μl saline via the intranasal route under i.v. ketamine anaesthesia (to prevent coughing). rEV131 was given at three dose levels (90, 180 and 360 μpg, 4.5-18 mg/Kg) to different groups of mice immediately before endotoxin by the same route, controls received saline only.

In a second set of experiments using this model, 350 μg budenoside (positive control), saline (negative control) and 182 μg rEV131 were given intraperitoneally by injection, one hour before the 1 μg LPS dose, which again was given by nasal inhalation.

Airways Resistance: Plethysmography

The airways resistance was evaluated by whole-body plethysmography for 3 h after endotoxin administration. After a recovery period bronchial hyperreactivity (BHR) to aerosolized methacholine was then investigated. Unrestrained conscious mice who had received endotoxin and either active or control medication were first placed in whole-body plethysmography chambers (Buxco Electronic, Sharon, Colo., USA). The mouse is placed in one of two barometric plethysmography chambers linked to suction pumps that ensure constant airflow. The animal is introduced into the first chamber separated from the second in which pressure corresponds to atmospheric pressure. Each compartment is linked to two parts of a differential pressure captor, which is itself connected to an electronic amplifier and signals are analyzed by software. This system allows the quantification of many parameters during successive respiratory cycles. Using this system bronchoconstriction was evaluated for three hours using Enhanced Respiratory Pause (Penh) as an indicator of airways resistance.

Penh can be conceptualised as the phase shift of the thoracic flow and the nasal flow curves; increased phase shift correlates with increased respiratory system resistance. Penh is calculated by the formula Penh=(Te/RT-1)×PEF/PIF, where Te is expiratory time, RT is relaxation time, PEF is peak expiratory flow, and PIF is peak inspiratory flow. Penh values correspond to the mean of eleven events (cycles) every five seconds during the observation period.

The experiment was terminated after 180 minutes and mice were allowed to recover by being ventilated with high oxygen concentration before being investigated for residual BHR.

In this phase of the experiment methacholine at 300 mM was aerosolised and introduced into the plethysmograph chambers for 20 seconds and mean airway bronchoconstriction readings, as assessed by Penh, were obtained over a 15-min period, which is the duration of methacholine induced BHR.

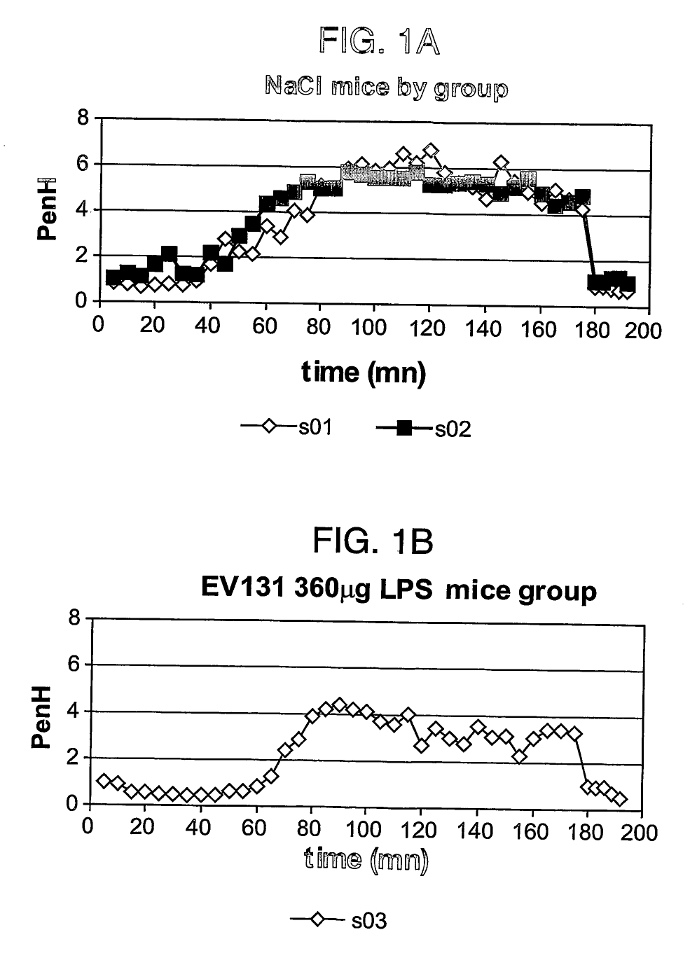

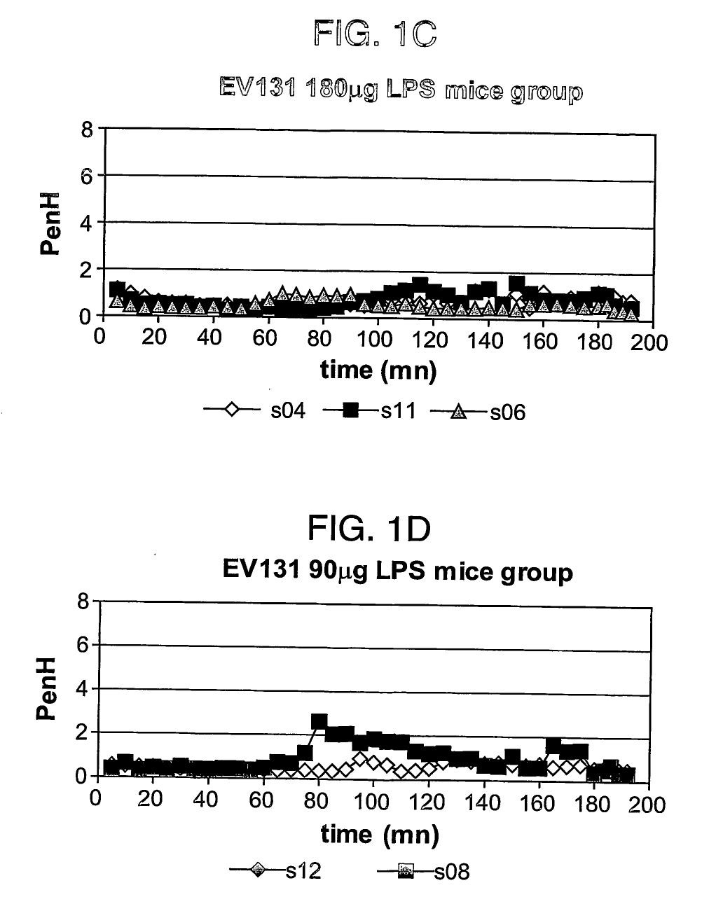

After analysis of data, Penh values are shown in FIG. 1 for 36 time points after endotoxin administration and 5 time points after methacholine nebulization. Penh values at every point correspond to the mean of Penh values between 5 min before and 3 min after the point. (NB In FIG. 1 the recovery period corresponds to the apparent drop in Penh at 180 minutes).

Bronchoalveolar Lavage (BAL)

BAL was performed under strong ketamine and xylasine anaesthesia 3.5 h after intranasal endotoxin administration by rinsing the airways with 4 volumes of 0.5 ml each of ice-cold phosphate buffered saline (PBS). The lavage fluid was centrifuged, resuspended, total cells were counted using a haematocytometer chamber and cytospin preparations were prepared using a Shandon cytocentrifuge. The cells were analysed: after differential staining with May-Gruenwald-Giemsa.

Myeloperoxidase Assay of the Lung (MPO)

In order to assess the neutrophil content in the lung we analysed the amount of myeloperoxidase, a major enzyme of neutrophils, in the lung as described before (also, see Hoy A, Leininger-Muller B, Kutter D, Siest G, Visvikis S. Clin Chem Lab Med 2002; 40(1): 2-8.)

Lung Histology

After bronchoalveolar lavage, the mice were killed. The whole lung was removed and fixed in 4% buffered formaldehyde for standard microscopic analysis using H&E stain. The peribronchial infiltrate and the smooth muscle hyperplasia was assessed by a semi-quantitative score (0-3) by two independent observers.

Results

Bronchoconstriction Induced by Endotoxin is Inhibited by EV131

Firstly we established a dose-response effect of endotoxin (1-100 μg) that induced non-lethal bronchoconstriction. Endotoxin was found to induce a substantial bronchoconstriction within 15-30 min (data not shown). We selected a dose of 1 μg of endotoxin for the further experiments in order to test the effect of rEV131.

In the control group LPS induced a substantial bronchoconstriction that peaked at about 80 minutes and persisted for 180 minutes until the mice were allowed to recover in high oxygen conditions. When given intranasally at a dose of 360 μg rEV131 partially inhibited this response whilst, 90 μg and 180 μg had a greater inhibitory effect (FIG. 1). This result was initially surprising, but subsequently, it was realised that the mouse given 360 μg rEV131 was suffering from an infection and probably a neutrophilia pre-treatment, and was thus not considered typical. Results from this mouse were excluded from subsequent analyses.

These data suggest that endogenous histamine plays a role in bronchoconstriction induced by endotoxin, and hence neutralisation of histamine by rEV131 could ameliorate ARDS.

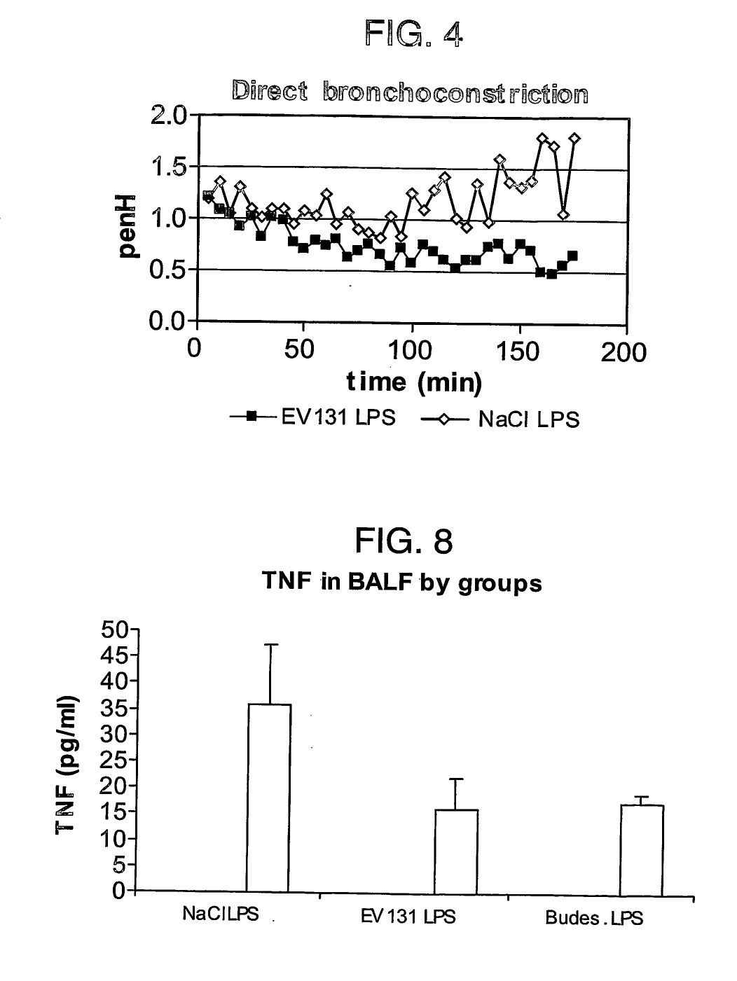

FIG. 4 shows a similar effect for rEV131 given intraperitoneally, proving that the rEV131 cannot be binding the LSP directly. This also demonstrates the rEV131 is effective when administered by this route.

Bronchial Hyperreactivity (BHR) is Inhibited by EV131

We also tested methacholine-induced BHR 3 h after endotoxin administration and recovery in high oxygen conditions. First we demonstrated that methacholine-mediated BHR occurs following endotoxin administration as compared to saline control (data not shown). After this we investigated the effect of methacholine in the rEV131 dosed and control mice. Methacholine provoked bronchoconstriction in control mice but not in mice treated with rEV131 at any dose level (FIG. 1). Therefore, the data suggest that endotoxin-induced hyperreactivity is histamine dependent and can be attenuated by rEV131.

Reduced Recruitment of Neutrophils in BAL and Lung

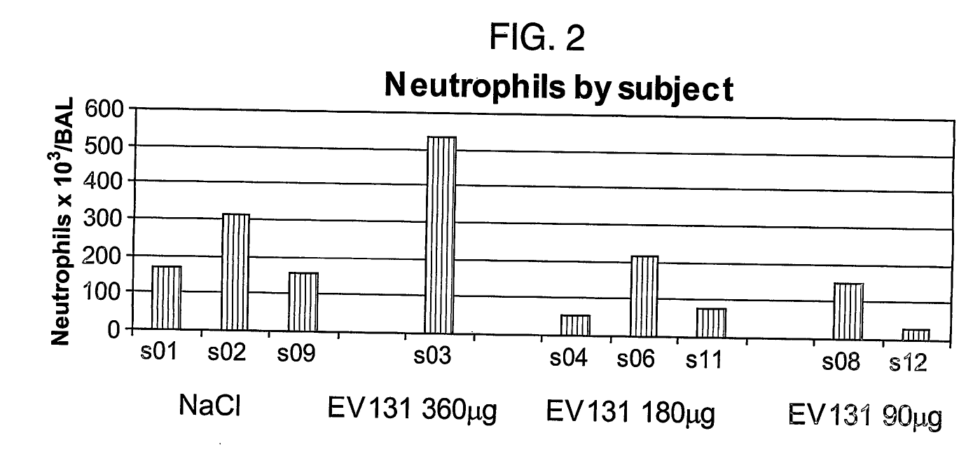

Administration of endotoxin results in a significant recruitment of neutrophils in BAL fluid. We recovered about 105 leukocytes in BAL fluid from control animals at 3 h after endotoxin inhalation. Administration of rEV131 did not alter the total cell count in the BAL fluid but, in contrast, the recruitment of neutrophils was reduced by rEV131 at 180 and 90 μg although this did not reach statistical significance (p<0.2). The 360 μg dose had no effect (FIG. 2); however, only one animal was evaluated at this dose and as stated above, this animal was subsequently identified as suffering from an infection.

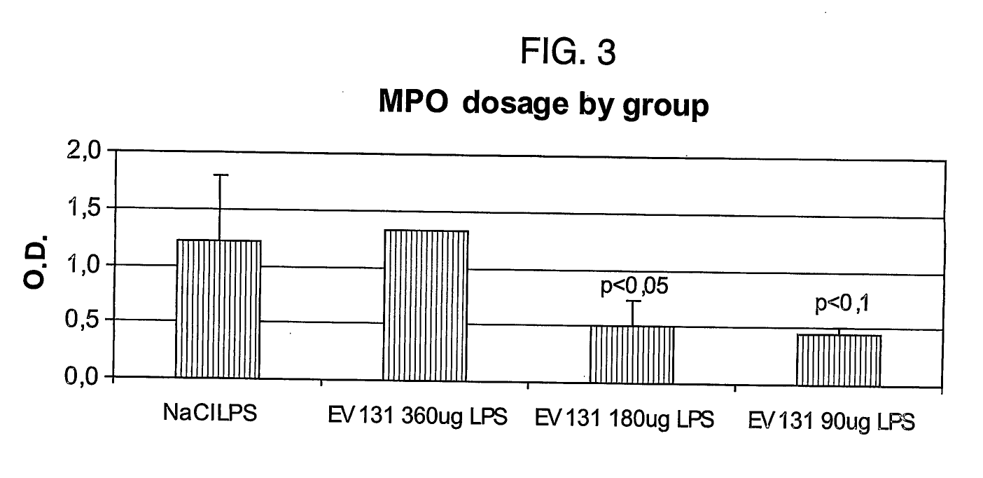

We also investigated whether the recruitment of activated neutrophils into the lungs was altered. In order to quantify the neutrophil recruitment we tested MPO activity of fresh lung homogenate. This showed a significant reduction of neutrophil activity by rEV131 at 180 μg (p<0.05) and 90 μg (p<0.01) (FIG. 3). In the infected mouse, administered 360 μg, there was no effect.

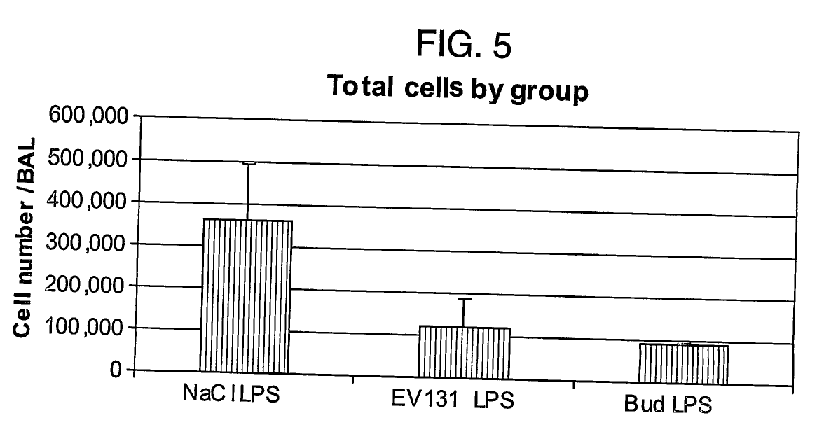

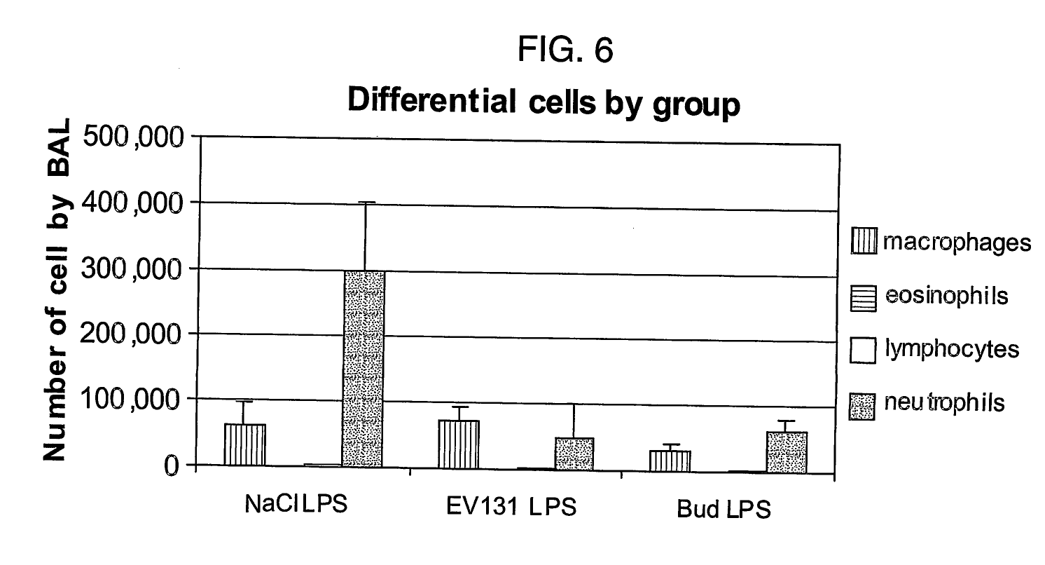

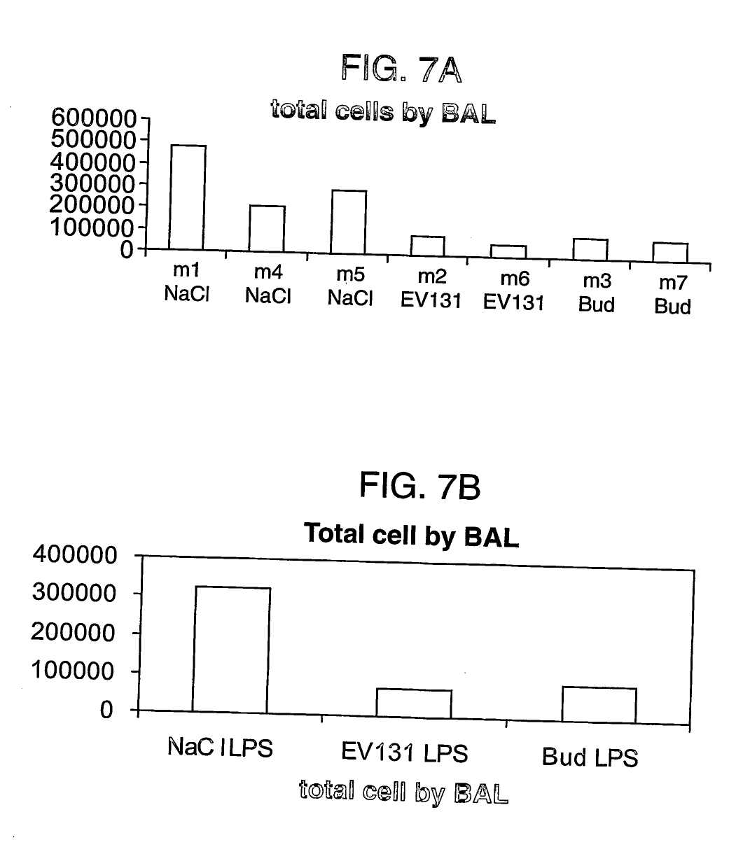

FIGS. 5, 6 and 7 are equivalent experiments performed to evaluate cell recruitment in BAL fluid when rEV131 and budenoside are given intraperitoneally. As is evident from these graphs, total cell numbers in BAL are significantly reduced by rEV131, and neutrophils in particular. Furthermore, the amount of TNF in the BAL fluid is also reduced by rEV131 (see FIG. 8).

Lung Histopathology:

Lungs from mice that received endotoxin showed significant peribronchial cellular infiltrates with abundant neutrophils (data not shown). rEV131 reduced the recruitment of neutrophils substantially at 180 μg and 90 μg doses (data not shown).

Conclusion

The present data demonstrate that the histamine binding protein rEV131 significantly inhibits endotoxin-induced bronchoconstriction, BHR and neutrophil recruitment in a murine model of ARDS. This effect is evident both when administered intranasally and intraperitoneally.

### EXAMPLE 2

### Reverse Passive Arthus Reaction

Methods

The classical reverse passive Arthus reaction was performed in mice, which represents a local immune complex pathology induced by the injection of antiserum in the skin followed by the intravenous injection of the antigen. The methods used were as follows:

Induction of Passive Arthus Reaction

C57/BL6 (129 or FVB) mice (6-8 weeks old, male and female) were shaved on the back. First, 25 μl of chicken anti-ovalbumin IgG (anti-Ova) (12.5-200 μg) or saline was injected intradermally in the back skin under isoflurane anaesthesia. Immediately thereafter, 100 μl ovalbumin (Ova) (1 mg containing 0.2% Evans blue) was injected into the tail vein. Control mice received intradermal injection of saline or bovine serum albumin or intravenous injection of saline of BSA (0.2% Evans blue).

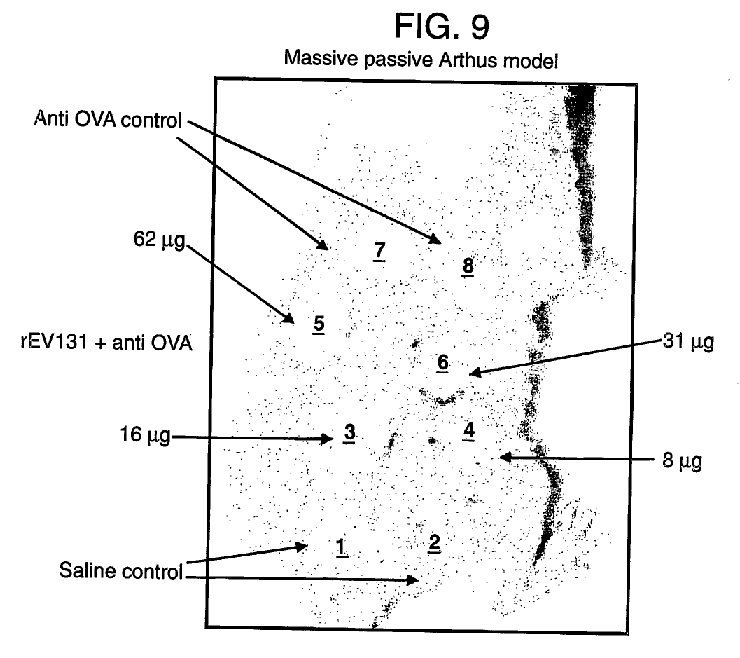

The scheme depicted in FIG. 9 describes the protocol used to test the inhibition of passive Arthus reaction by EV proteins: optimal quantity of anti-ovalbumin IgG (25 μg) for passive Arthus inhibition or saline was injected intradermally with or without test proteins.

Quantification of the Vascular Leak

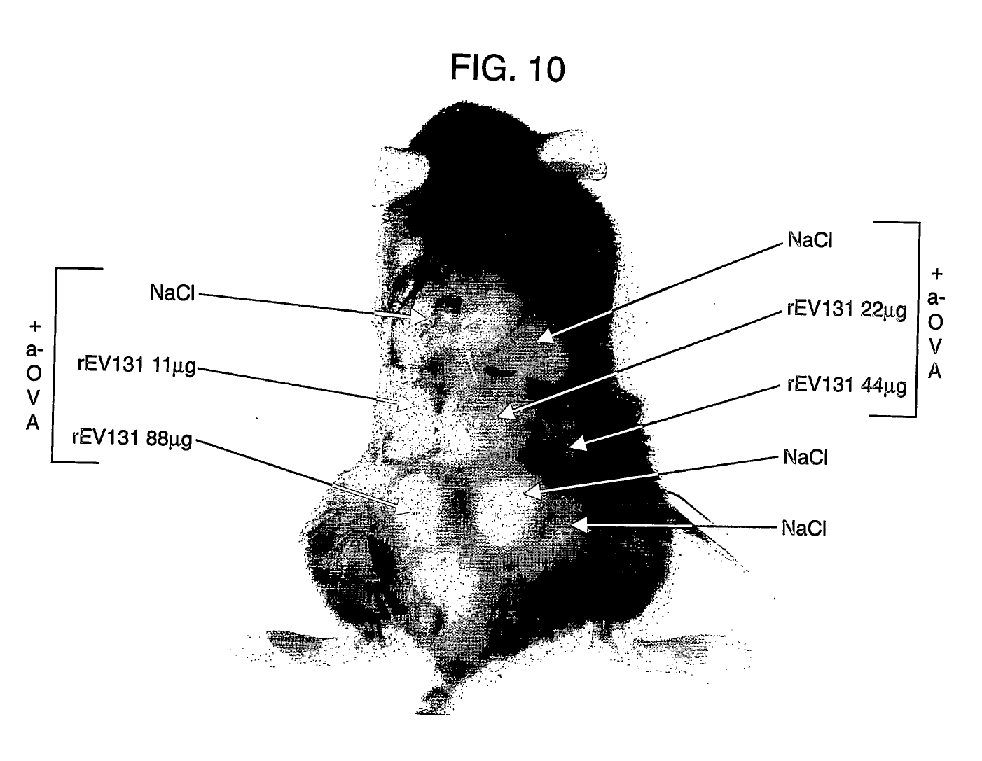

The formation of immune complexes and their local deposition induces an acute inflammatory response. As sign of plasma proteins extravasation consecutive to vasodilatation and endothelial damage, a blue discoloration is seen within 15 min at the intradermal injection site of anti-Ova serum of mice receiving an i.v. injection of Ova containing 0.2% Evans blue. The discolorations were recorded with a digital camera (FIG. 10).

The injection site was excised at 30 min, minced with scissors and digested in formamide overnight, and the absorption was measured by ELISA plate reader at OD 610 nm.

Evaluation of Cellular Reaction at the Injection Site by Microscopy

The injection site was excised at 6 h post-injection, fixed in 4% buffered formaldehyde, embedded in paraffin, cut at 5 μm on a Leica microtome, stained with H&E and analysed semiquantitatively by microscopically using a scoring system (0, no infiltration, 1, minimal, 2, moderate and 3 severe infiltration by polymorphonuclear neutrophils, PMN).

Results

Vascular Leakage

Positive controls. In the absence of test proteins the injection site of the skin stained deep blue within 15 min indicating vascular leak (results not shown).

Negative controls. Intravenous injection of saline (no antigen) containing 0.2% Evans blue did not cause any vascular leak, e.g. no blue discoloration (results not shown).

Inhibition of Vascular Leak by EV131 and EV504.

The test proteins were injected either alone or together with anti-Ova antibody (12.5 μg by site) into the skin. When injected with the anti-Ova antibody, there was an almost complete protection by EV131, and only a partial protection by EV504 (not shown). The estimated IC₅₀ is in the range of 16 and 31 μg for EV131 and EV504 respectively. The results from these experiments are highly reproducible. By contrast, the intradermal injection of the test proteins alone, did not cause any vascular leak (data not shown).

Dose Dependence of Inhibition by EV131 and EV504.

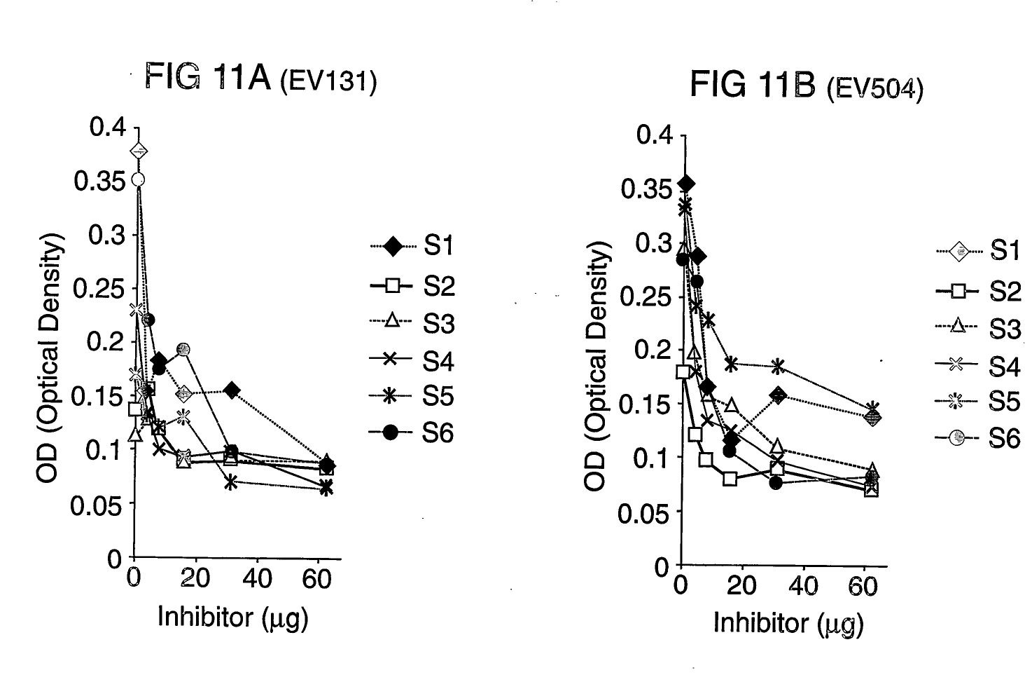

The dose dependence of the inhibitory effects was quantified by spectrophotometry of the excised and digested skin. The IC₅₀ for EV131 and EV504 were in the range of 20 μg and 60 μg, respectively (FIG. 11).

Neutrophil Infiltration

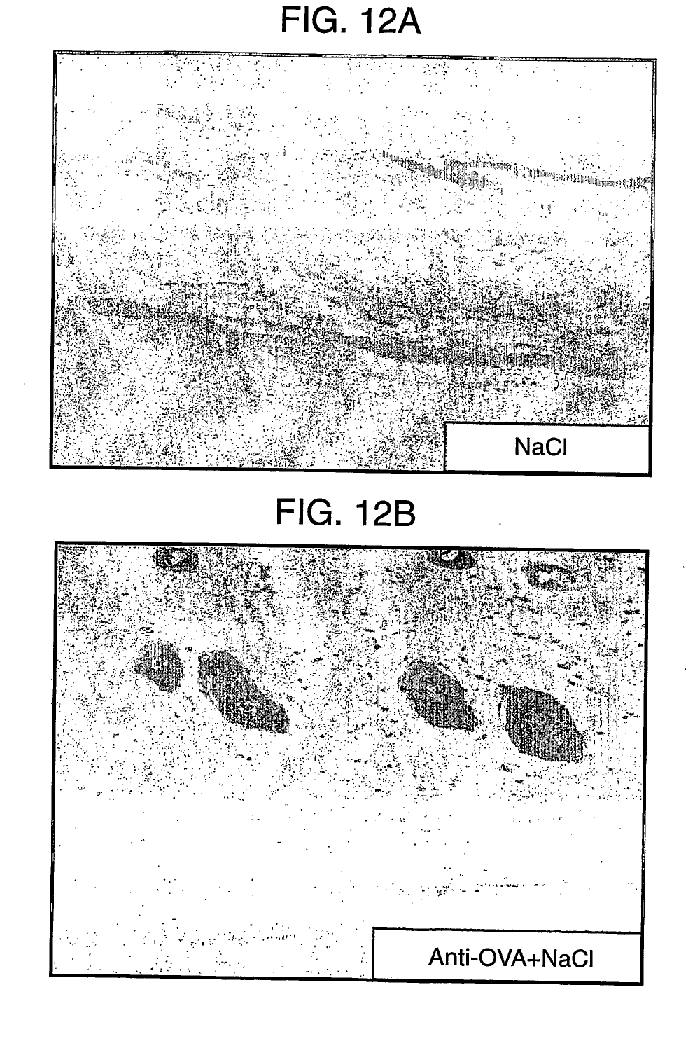

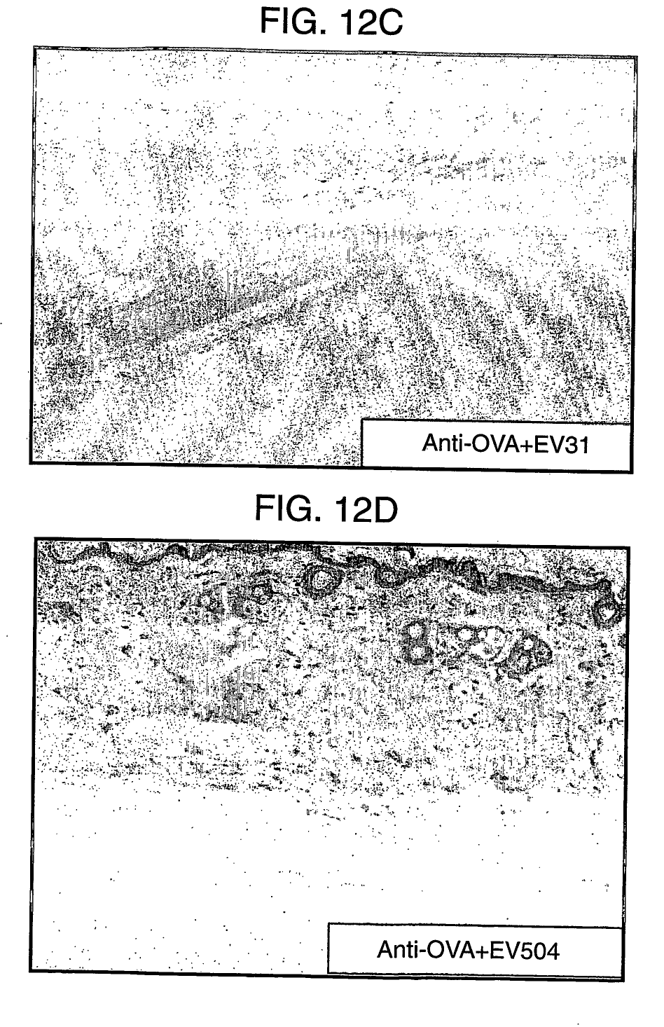

The dermal injection site was excised at 6 h postinjection of anti-Ova and processed for histology. The dermis of anti-Ova injected control mice showed distinct perivascular neutrophil infiltrations. There was no infiltrate found in the saline injected controls (FIG. 12). EV131 and to a lesser extent EV504 reduced significantly the immune complex induced recruitment of PMN into the skin (62.5 μg). For accurate quantification, the experiments need to be repeated due to some technical failures during the skin sampling. The results of this experiment are summarised in Table 1 below.

Therefore, both test proteins have an inhibitory effect on the early vascular leakage in the reverse Arthus reaction. EV131 appears to be more potent than EV504 in this immune complex model in the mouse. The IC₅₀ for the inhibition of the vascular leak was at 20 μg and 60 μg for EV131 and EV504, respectively (FIG. 11). At high doses the infiltration of PMN was almost abolished.

TABLE 1
PMN infiltration (Reverse Arthus Reaction)

| Injection | | | | |
|---|---|---|---|---|
| | | Quantity | | PMN Infiltration |
| n^{o} | Products | EV proteins (μg/site) | Anti-Ova (μg/site) | score |
| 10 | Nacl | 0 | 0 | − |
| 10 | a-Ova + Nacl | 0 | 25 | ++ |
| 4 | a-Ova + EV131 | 85 | 25 | − |
| 4 | a-Ova + EV504 | 150 | 25 | − |
| 2 | a-Ova + EV504 | 75 | 25 | + |
| 4 | a-Ova + control protein | 62 | 25 | ++ |

### EXAMPLE 3

### Allergic Conjunctivitis

A study was performed to evaluate the safety and efficacy of FS-HBP2 (rEV131) in the prevention of the signs and symptoms of allergic conjunctivitis as induced by the conjunctival allergen challenge model (Abelson M B, Chambers W A and L M Smith; Ophthalmology, 1990; 108:84-88). Four treatments were applied, involving a comparison of the rEV131 vehicle against three concentrations of rEV131 (0.06%, 0.12% and 0.24% ophthalmic solutions). Sixty subjects enrolled in the study.

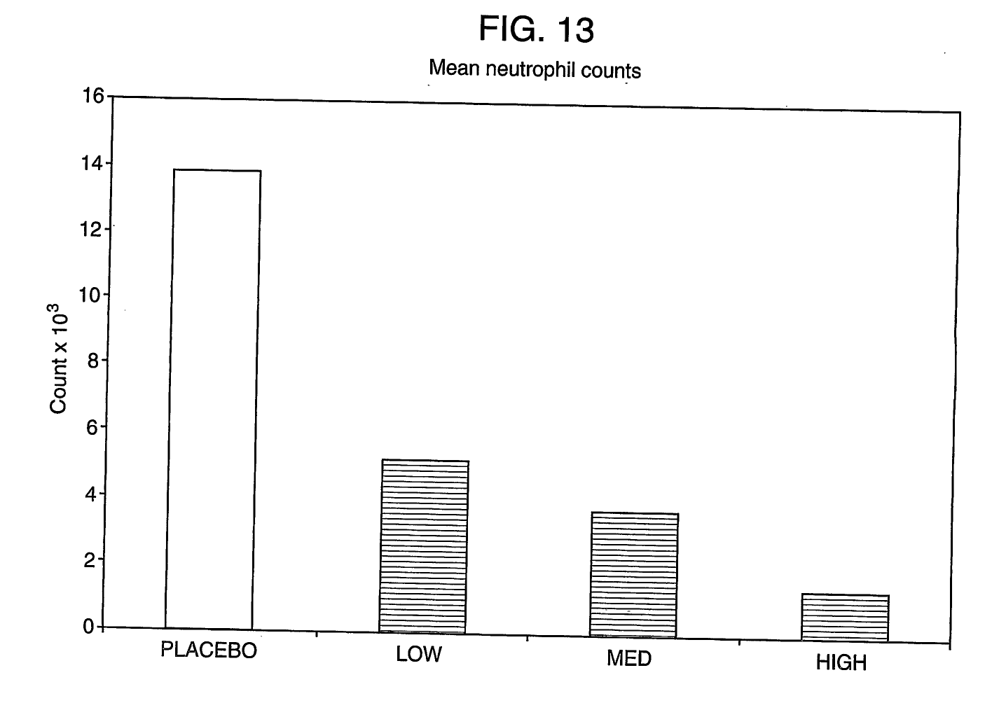

Primary efficacy variables that were measured included ocular itching and redness. As part of the secondary efficacy variables measured, neutrophil counts were assessed. To do this, tear samples were collected from 23 subjects who participated in the study. Of those, 19 subjects had detectable neutrophil counts. In subjects that received the 0.12% (N=3) and 0.24% (N=7) concentrations of rEV131 in one eye and placebo in the fellow eye, neutrophil counts were significantly less in the drug-treated eye (see FIG. 13). However, in the group that received 0.06% rEV131 in one eye and placebo in the other eye, neutrophil counts were significantly greater in the eyes that received medication. In the eyes receiving vehicle bilaterally (N=5), no significant difference in neutrophil counts were found.

These results suggest that unpreserved rEV131 may significantly decrease the number of neutrophils recruited to the eye during or immediately after the conjunctival allergen challenge.

These results, when combined with the results presented in Examples 1 and 2, suggest that unpreserved rEV131 (i.e. rEV131 solutions that do not contain the preservative benzalkonium chloride, with which the protein is suspected to complex) may play a more significant role in decreasing inflammation and subsequent tissue damage associated with more specifically, neutrophil-mediated diseases, in the acute allergic reaction in the eye. The late phase allergic reaction is mediated by the infiltrate of leukocytes into the tissue via chemotactic factors released by the mast cell during the early phase acute reaction. In the eye, while there may be a physiologic late phase with cellular infiltrate, most cases of allergic conjunctivitis do not have a clinically relevant late phase. In the eye, only severe, chronic allergic reactions consist of a late phase reaction which reaches a certain threshold and induces clinical signs and symptoms, such as keratitis and shield ulcers seen in vernal keratoconjunctivitis. However, the nose and lung do manifest clinical late phase reactions more prominently than the eye. This may explain the effects of rEV131 seen on nasal symptoms induced by conjunctivial allergen challenge (CAC) in a previous clinical study. An allergic reaction in the nose, following CAC, and effects of an agent instilled in the eye, on nasal symptoms, is not unexpected since allergen, mediators, and active drug products, can all drain from the ocular surface, through the nasolacrimal ducts, into the inferior turbinate of the nasal cavity where it can elicit effects on nasal tissues.

Subsequent studies will focus on the potential of histamine binding molecules such as rEV131 to reduce specifically neutrophil-mediated reactions, such as post-operative inflammation or marginal infiltrates.

1. A method of treating a disease condition mediated by neutrophil cells in a patient, comprising administering a histamine binding compound to the patient in a therapeutically-effective amount.

2. A method according to claim 1, wherein said disease condition is an allergic condition, an inflammatory condition or an auto-immune condition.

3. A method according to claim 1, wherein said disease condition is selected from the group consisting of adult respiratory distress syndrome (ARDS); infant respiratory distress syndrone (IRDS); severe acute respiratory syndrome (SARS); chronic obstructive airways disease (COPD); cystic fibrosis; ventilator induced lung injury (VILI); capillary leak syndrome; reperfusion injury including injury following thrombotic stroke, coronary thrombosis, cardiopulmonary bypass (CPB), coronary artery bypass graft (CABG), limb or digit replantation, organ transplantation, post-operative inflammation or marginal infiltrates, bypass enteritis, bypass arthritis, thermal injury and crush injury; psoriasis; psoriatic arthropathy; rheumatoid arthritis; Crohn's disease; ulcerative colitis; immune vasculitis including Wegener's granulomatosis and Churg-Strauss disease; alcoholic liver disease; neutrophil mediated glomerulonephritis; systemic lupus erythematosus; lupus nephritis; atherosclerosis; systemic sclerosis; gout; periodontal disease, ocular inflammation including dry eye, Sjogren's syndrome, contact lens associated papillary conjunctivitis (CLAPC), contact lens associated marginal infiltrates, post surgical inflammation including surgery for cataract, glaucoma, corneal transplantation and laser in-situ keratomileusis (LASIK), severe allergic conjunctivitis, vernal keratoconjunctivitis (VKC), diffuse lamellar keratitis, infective and non-specific conjunctivitis, keratitis and blepharitis, shield ulcers.

4. A method according to claim 1, wherein said histamine binding compound is a histamine scavenger.

5. A method according to claim 4, wherein said histamine scavenger binds to histamine with a dissociation constant of greater than 10⁻⁷M.

6. A method according to claims 1, wherein said histamine binding acompound is a protein.

7. A method according to claim 6, wherein said histamine binding protein is a vasoactive amine binding protein.

8. The method according to claim 1, where the vasoactive amine binding protein is:
a) any vasoactive amine binding protein that binds specifically to histamine with a dissociation constant of less than 10⁻⁷M and which belongs to the same protein family as the proteins MS-HBP1, FS-HBP1 and FS-HBP-2 disclosed in International Patent Application No. WO97/44451, wherein a protein is considered to belong to this protein family if the primary, mature monomer sequence of the protein has no more than 260 amino acids and at least 30 of the amino acids in the protein's complete sequence, preferably at least 40, 50, 60, 70, 80, 90, 100 or more, are conserved as identical residues in an alignment of that protein and the proteins MS-HBP1, FS-HBP 1 and FS-HBP-2, the alignment preferably having been obtained using ClustalW (Thompson et al., 1994, NAR, 22(22), 4673-4680);
b) a protein from a haematophagous arthropod that binds specifically to histamine with a dissociation constant less than 10⁻⁷M and which contains the sequence motifs D/E A W K/R (preferably DAWK, more preferably QDAWK) and Y/C E/D L/I/F W (preferably Y/C ELW);
c) a natural biological variant, such as an allelic variant or a geographical variant, of a protein as defined in (a) or (b) above;
d) a functional equivalent of a protein as defined in (a), (b) or (c) above that contains single or multiple amino-acid substitution(s), addition(s), insertion(s) and/or deletion(s) from the wild type protein sequence and/or substitutions of chemically-modified amino acids that do not affect the biological function of binding to histamine;
e) an active fragment of a protein as defined in (a), (b), (c) or (d) above, wherein “active fragment” denotes a truncated protein that retains the biological function of binding to histamine; and
f) a fusion protein comprising a protein as defined in (a), (b), (c), (d) or (e) above fused to a peptide or other protein, such as a label, which may be, for instance, bioactive, radioactive, enzymatic or fluorescent, or an antibody.

9. A method according to claim 7, wherein said vasoactive amine binding protein is EV131 or a fragment thereof.

10. (canceled)
